(19) United States
(12) Patent Application Publication
(10) Pub. No.: US 2013/0244897 A1
(43) Pub. Date: Sep. 19, 2013

(54) Marker Sequences for Multiple Sclerosis and Use Thereof

(71) Applicants: Angelika Lueking, Bochum (DE); Axel Kowald, Bochum (DE); Heike Göhler, Bochum (DE)

(72) Inventors: Angelika Lueking, Bochum (DE); Axel Kowald, Bochum (DE); Heike Göhler, Bochum (DE)

(73) Assignee: Protagen AG, Dortmund (DE)

(21) Appl. No.: 13/877,248

(22) PCT Filed: Oct. 4, 2011
(86) PCT No.: PCT/EP2011/067285

§ 371 Date: May 31, 2013

(30) Foreign Application Priority Data
Oct. 1, 2010 (EP) 10186327.2

(51) Int. Cl. C12Q 1/68; G01N 33/68

(52) U.S. Cl. CPC C12Q 1/6883; G01N 33/6854
USPC 506/9; 436/501; 506/16; 506/18; 210/646; 604/6.01

(57) ABSTRACT

The invention relates to novel marker sequences for multiple sclerosis and to the use thereof in diagnosis as well as to a method for screening potential active ingredients for multiple sclerosis diseases the marker sequences. The invention further relates to a diagnostic device containing such marker sequences for multiple sclerosis, especially to a protein biochip and the use thereof.

The present invention relates to novel marker sequences for multiple sclerosis and to the use thereof in diagnosis, together with a method for screening potential active ingredients for multiple sclerosis diseases by way of these marker sequences. The invention further relates to a diagnostic device comprising such marker sequences for multiple sclerosis, in particular a protein biochip, and to the use thereof.

Protein biochips are gaining increasing industrial importance for analytical and diagnostic purposes as well as in pharmaceutical development. Protein biochips have also become established as screening tools.

To this end, the fast and highly parallel detection of a plurality of specifically binding analysis molecules during a single experiment is made possible. Producing protein biochips requires the availability of the necessary proteins. For this purpose, in particular protein expression libraries have become established. High throughput cloning of defined open reading frames is one option (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P. (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Vaglio, P., Rual, J. F., Lamesch, P., Martinez, M., Armstrong, C. M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, such an approach is highly dependent on the progress of genome sequencing projects and the annotation of these gene sequences. Moreover, the determination of the expressed sequence can be ambiguous due to differential splicing processes. This problem can be circumvented by the use of cDNA expression libraries (Büssow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, Nordhoff, E., Lübert, C., Lehrach, H. and Walter, G. (2000) A human cDNA library for high-throughput protein expression screening. Genomics, 65, 1-8; Holz, C., Lueking, A., Bovekamp, L., Gutjahr, C., Bolotina, N., Lehrach, H. and Cahill, D. J. (2001) A human cDNA expression library in yeast enriched for open reading frames. Genome Res, 11, 1730-1735; Lueking, A., Holz, C., Gotthold, C., Lehrach, H. and Cahill, D. (2000) A system for dual protein expression in Pichia pastoris and Escherichia coli, Protein Expr. Purif., 20, 372-378). To this end, the cDNA of a particular tissue is cloned into a bacterial or eukaryotic expression vector, such as yeast. The vectors used for expression are generally characterized in that these carry inducible promoters, by way of which the time of protein expression can be controlled. In addition, expression vectors comprise sequences for so-called affinity epitopes or affinity proteins, which permit the specific detection of recombinant fusion proteins by way of an antibody that is directed against the affinity epitope and additionally enable specific purification by way of affinity chromatography (IMAC).

For example, the gene products of a cDNA expression library from human fetal brain tissue in the bacterial expression system Escherichia coli were arranged in a high-density format on a membrane and able to be successfully screened with various antibodies. It was shown that the proportion of full-length proteins was at least 66%. It was further possible to express the recombinant proteins from expression libraries in high throughput and purify them (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Buessow (2000) supra; Lueking, A., Horn, M., Eickhoff, H., Buessow, K., Lehrach, H. and Walter, G. (1999) Protein microarrays for gene expression and antibody screening. Analytical Biochemistry, 270, 103-111). Such cDNA expression library-based protein biochips are the subject matter in particular of WO 99/57311 and WO 99/57312.

In addition to antigen-presenting protein biochips, antibody-presenting arrangements are described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

However, there is a high need to make indication-specific diagnostic devices, such as a protein biochip, available.

The object of the present invention is to provide improved marker sequences and the diagnostic use thereof for treating multiple sclerosis.

The provision of specific marker sequences allows a reliable diagnosis and stratification of patients with multiple sclerosis, in particular by way of a protein biochip.

The invention therefore relates to the use of marker sequences for diagnosing multiple sclerosis, wherein at least one marker sequence of a cDNA selected from the group SEQ 1-308, or a respective protein coding therefor, or a respective partial sequence or fragment thereof (hereinafter: marker sequences according to the invention) is determined on or from a patient to be examined.

It was possible to identify the marker sequences according to the invention by way of differential screening of samples, specifically from healthy participants, with samples from patients with multiple sclerosis.

For the first time, these marker sequences according to the invention were identified by way of protein chips (see examples).

In the prior art, marker sequences for multiple sclerosis were already identified using a protein biochip, see WO2009030225. However, in the present case according to the invention, improved bioinformational evaluation is aspired, and the samples are particularly preferably taken from the cerebrospinal fluid (CSF). Moreover, specifically selected samples are used, which accommodate the high sensitivity of a protein biochip.

The term “multiple sclerosis ((MS), also encephalomyelitis disseminata)” relates to an autoimmune inflammatory/demyelinating and degenerative disease of the central nervous system (for example, definition according to Pschyrembel, de Gruyter, 261st edition (2007), Berlin).

It is essential for the invention that the samples are not taken from conventional blood banks, but were carefully selected from MS patients who are HIV and HCV negative, for example, and were tested in particular for infectious diseases. The complex sample selection procedure allows, for example, sufficient advantageous differentiation from diseases such as neuroborreliosis with symptoms similar to MS. Moreover, false positive results are excluded, for one because of the strict bioinformational evaluation (see examples), and secondly by comparing the results on a protein chip according to the invention to, for example, sera of neuroborreliosis patients without multiple sclerosis.

Contrary to WO2009030225, the protein biochips are additionally produced by normalizing at least 1,000, preferably 2,000 different, or more, autoantigens of humans, which are not indication-specific of multiple sclerosis. For example, such autoantigens can be obtained from other bodily fluids of patients with other illnesses (such as pancreatic cancer, rheumatoid arthritis, prostate and the like).

The invention therefore also relates to such indication-specific protein biochips according to the invention for diagnosing multiple sclerosis, wherein in a further step, the proteins or marker sequences represented on the protein biochip are normalized with autoantibodies from non-multiple sclerosis patients and false positive proteins can be eliminated in this way. Remaining non-false positive proteins can be newly arranged on a protein biochip, which is referred to as rearraying. This likewise allows autoantibodies with a positive response to E. coli to be excluded. This is a further qualitative improvement, for example because autoantibodies that are directed to E. coli enterobacteria in humans can be excluded. As a result, new marker sequences can advantageously be identified with an improved signal-to-noise ratio.

In a further preferred embodiment, at least 2 to 5 or 10, preferably 30 to 50 marker sequences, or 50 to 100 or more marker sequences are thus determined on or from a patient to be examined.

In a further embodiment of the invention, the marker sequences according to the invention can also be combined, supplemented or expanded with known biomarkers for this indication.

In a preferred embodiment, the marker sequences are determined outside the human body and the determination is carried out in an ex vivo/in vitro diagnosis.

In a further embodiment of the invention, the invention relates to the use of marker sequences as diagnostic products, wherein at least one marker sequence of a cDNA is selected from the group SEQ 1-308, or a respective protein coding therefor, or a respective partial sequence or fragment thereof.

The invention further relates to a method for diagnosing multiple sclerosis, wherein a.) at least one marker sequence of a cDNA selected from the group SEQ 1-308, or a protein coding therefor, or a respective partial sequence or fragment thereof, is applied to a solid support, and b.) brought in contact with body fluid or tissue extract and c,) the detection of an interaction of the body fluid or tissue extract with the marker sequences from a.) is carried out.

The invention therefore also relates to diagnostic products for diagnosing multiple sclerosis, each selected from the group SEQ 1-308, or a respective protein coding therefor, or a respective partial sequence or fragment thereof.

Such an interaction can be detected by a probe, in particular by an antibody, for example.

The invention therefore also relates to the problem of providing a diagnostic device or an array, in particular a protein biochip, which allows a diagnosis of or examination for multiple sclerosis.

The invention further relates to a method for the stratification, in particular for the risk stratification, and/or treatment management of a patient with multiple sclerosis, wherein at least one marker sequence of a cDNA selected from the group SEQ 1-308, or a respective protein coding therefor, is determined on a patient to be examined.

The invention further encompasses the stratification of patients with multiple sclerosis into new or established sub-groups of multiple sclerosis as well as the expedient selection of patient groups for the clinical development of new therapeutic agents. The term treatment management also includes dividing the patients into responders and non-responders with respect to a treatment or the treatment course thereof.

The term “diagnosis” within the meaning of the present invention denotes the positive identification of multiple sclerosis by way of the marker sequences according to the invention and the association of patients with the multiple sclerosis disease. The term ‘diagnosis’ comprises medical diagnostics and examinations in this regard, in particular in vitro diagnostics and laboratory diagnostics, as well as proteomics and nucleic acid blotting. Additional examinations may be required for validation and to exclude other illnesses. The term ‘diagnosis’ therefore likewise encompasses the differential diagnosis of multiple sclerosis by way of the marker sequences according to the invention and the prognosis of multiple sclerosis.

“Stratifying (also: stratification) or treatment management” within the meaning of the present invention shall mean that the method according to the invention allows decisions regarding the treatment and therapy of the patient, be it hospitalization of the patent, use, effect and/or dosage of one or more pharmaceuticals, a therapeutic measure or monitoring the progression of an illness or treatment, or etiology or classification of a disease, for example into a new or existing sub-type, or the differentiation of illnesses and the patients thereof.

In a further embodiment of the invention, the term “stratification” comprises in particular risk stratification with the prognosis of an outcome of a disadvantageous health event.

Within the scope of the present invention, the term “patient” is considered to mean any participant—human or mammal—with the proviso that the participant is examined for multiple sclerosis.

The term “marker sequences” within the meaning of the present invention shall mean that the cDNA, or the respective polypeptide or protein obtainable therefrom, is significant for multiple sclerosis. For example, the cDNA, or the respective polypeptide or protein obtainable therefrom, can exhibit an interaction with substances from the body fluid or tissue extract of a patient with multiple sclerosis (for example antigen (epitope)/antibody (paratope) interaction). Within the meaning of the invention, “wherein at least one marker sequence of a cDNA selected from the group SEQ 1-308, or a respective protein coding therefor, or a respective partial sequence or fragment thereof, is determined on a patient to be examined” shall mean that an interaction between the body fluid or tissue extract of a patient and the marker sequences according to the invention is detected. Such an interaction includes, for example, a bond, in particular a binding substance on at least one marker sequence according to the invention or, in the case of cDNA, hybridization with a suitable substance under select conditions, in particular stringent conditions (for example as customarily defined in J. Sambrook, E. F. Fritsch, T. Maniatis (1989), Molecular cloning: A laboratory manual, 2nd Edition, Cold Spring Habor Laboratory Press, Cold Spring Habor, USA or Ausubel, “Current Protocols in Molecular Biology”, Green Publishing Associates and Wiley Interscience, N.Y. (1989)). One example of stringent hybridization conditions is: hybridization in 4×SSC at 65° C. (alternatively in 50% formamide and 4×SSC at 42° C.), followed by several washing steps in 0.1×SSC at 65° C. for a total of approximately one hour. One example of less stringent hybridization conditions is hybridization in 4 s SCC at 37° C., followed by several washing steps in 1×SCC at room temperature.

According to the invention, such substances are constituents of a body fluid, in particular blood, whole blood, blood plasma, serum, patient serum, urine, cerebrospinal fluid, synovial fluid or a tissue extract of the patient.

In a further embodiment of the invention, however, the marker sequences according to the invention can be present in a significantly higher or lower expression rate or concentration, indicating multiple sclerosis. To this end, the relative sick/healthy expression rates of the marker sequences for multiple sclerosis according to the invention are determined by way of proteomics or nucleic acid blotting.

In a further embodiment of the invention, the marker sequences comprise a detection signal that is addressed to the substance to be bound (for example antibody, nucleic acid). According to the invention, the detection signal is preferably an epitope and/or paratope and/or haptene for a protein, and it is a hybridization region or binding region for a cDNA.

The marker sequences according to the invention are listed in Table A and can be unambiguously identified by the respective cited database entry (also via the Internet: http://www.ncbi.nlm.nih.gov/) (see in Table A: Accession No. there), see also the associated sequence protocol.

The invention thus likewise relates to full-length sequences of the markers according to the invention, more particularly as defined in Table 1 by way of the known database entry, hereafter referred to as SEQ 1a-308a (cDNA) and SEQ 1b-308b (protein).

The invention therefore also comprises embodiments of SEQ 1a-308a that are analogous to the marker sequences SEQ 1-308, as described in the claims for example, because the SEQ 1-308 according to the invention again represent partial sequences, at least with high homology. However, the specific marker sequences SEQ 1-308 are preferred according to the invention.

According to the invention, the marker sequences also comprise modifications of the cDNA sequence, and of the corresponding amino acid sequence, such as chemical modification, for example citrullination, acetylation, phosphorylation, glycosylation or polyA tail and other relevant modifications known to a person skilled in the art.

Another embodiment of the invention also encompasses partial sequences or fragments of the marker sequences according to the invention. These are in particular partial sequences that are 95%, 90%, notably 80% or 70% identical to the marker sequences according to the invention.

Partial sequences also include sequences that comprise 50 to 100 nucleotides, 70 to 120 nucleotides of a sequence of SEQ 1-308, or peptides obtainable therefrom.

“Partial sequences or fragments” of the marker sequences according to the invention are functionally defined and comprise sequences that have the same diagnostic function according to the invention.

In a further embodiment, the respective marker sequence may be represented in differing quantities in one or more regions on a solid support. This allows the sensitivity to be varied. The regions can each comprise a collectivity of marker sequences, which is to say a sufficient number of different marker sequences, in particular 2 to 5, or 10 or more, and optionally additional nucleic acids and/or proteins, in particular biomarkers. However, at least 96 to 25,000 (numerical) or more different or identical marker sequences and additional nucleic acids and/or proteins, in particular biomarkers, are preferred. Further preferred are more than 2,500, particularly preferred are 10,000 or more different or identical marker sequences and optionally additional nucleic acids and/or proteins, in particular biomarkers.

Another object of the invention is an arrangement of marker sequences comprising at least one marker sequence of a cDNA selected from the group SEQ 1-308, or a respective protein coding therefor. The arrangement preferably comprises at least 2 to 5 or 10, preferably 30 to 50 marker sequences, or 50 to 100 or more marker sequences.

Within the scope of the present invention, “arrangement” shall be synonymous with “array”, and provided that this “array” is used to identify substances on marker sequences, this shall be understood to mean an “assay” or a diagnostic device. In a preferred embodiment, the arrangement is designed so that the marker sequences represented on the arrangement are present in the form of a grid on a solid support. Moreover, arrangements that allow a high-density arrangement of protein binders and where the marker sequences are spotted are preferred. Such high-density spotted arrangements are disclosed in WO 99/57311 and WO 99/57312, for example, and can advantageously be employed in a robot-assisted automated high-throughput method.

However, within the scope of the present invention the term “assay” or diagnostic device also comprises embodiments of a device, such as ELISA, bead-based assay, line assay, western blot, immunochromatographic methods (for example so-called lateral flow immunoassays) or similar immunological single or multiplex detection methods. A protein biochip within the meaning of the present invention is a systematic arrangement of proteins on a solid support.

The marker sequences of the arrangement are fixed on a solid support, however preferably they are spotted or immobilized, even printed on, which is to say they are applied reproducibly. One or more marker sequences can be present multiple times in the collectivity of all marker sequences and be present in differing quantities relative to a spot. Furthermore, the marker sequences can be standardized on the solid support (for example by way of human globulin serial dilution series as internal calibrators for data normalization and quantitative evaluation).

As a result, the invention also relates to an assay or a protein biochip comprising an arrangement containing marker sequences according to the invention.

In a further embodiment, the marker sequences are present in the form of clones. For example, such clones can be obtained by way of a cDNA expression library according to the invention (Büssow et al. 1998 (supra)). In a preferred embodiment, such expression libraries containing clones are obtained by way of expression vectors from an expressing cDNA library comprising the cDNA marker sequences. These expression vectors preferably comprise inducible promoters. The expression can, for example, be induced by way of an inducer such as IPTG. Suitable expression vectors are described in Terpe et al. (Terpe T Appl Microbiol Biotechnol. 2003 January; 60(5):523-33).

Expression libraries are known to a person skilled in the art and can be produced according to standard reference books such as Sambrook et al, “Molecular Cloning, A laboratory handbook, 2nd edition (1989), CSH press, Cold Spring Harbor, N.Y. Also preferred are expression libraries that are tissue-specific (for example human tissue, in particular human organs). According to the invention, expression libraries that can be obtained by way of exon trapping are also covered. The term ‘expression bank’ can be employed synonymously for the term expression library.

Also preferred are protein biochips or corresponding expression libraries that have no redundancy (so-called: Uniclone® library) and that can be produced according to the teachings of WO 99/57311 and WO 99/57312, for example. These preferred Uniclone libraries have a high proportion of non-defective fully expressed proteins of a cDNA expression library.

Within the scope of the present invention, the clones can also be, but are not limited to, transformed bacteria, recombinant phages or transformed cells from mammals, insects, fungi, yeast or plants.

The clones are fixed, spotted or immobilized on a solid support.

The invention thus relates to an arrangement, wherein the marker sequences are present in the form of clones.

The marker sequences can also be present in the form of a fusion protein comprising at least one affinity epitope or “tag”, for example. The tag may be one such as c-myc, his tag, arg tag, FLAG, alkaline phosphatase, V5 tag, T7 tag or strep tag, HAT tag, NusA, S tag, SBP tag, thioredoxin, DsbA, including a fusion protein, preferably a cellulose-binding domain, green fluorescent protein, maltose-binding protein, calmodulin-binding protein, glutathione S-transferase or lacZ.

In all embodiments, the term “solid support” encompasses designs such as a filter, a membrane, a magnetic or fluorophore-labeled bead, a silicon wafer, glass, metal, plastic material, a chip, a mass spectrometry target or a matrix. However, a filter is preferred according to the invention.

Moreover, PVDF, nitrocellulose or nylon are preferred filters (for example Immobilon P Millipore, Protran Whatman, Hybond N+ Amersham).

In a further preferred embodiment of the arrangement according to the invention, this arrangement corresponds to a grid having the size of a microtiter plate (8-12 wells, 96 wells, 384 wells or more), a silicon wafer, a chip, a mass spectrometry target or a matrix.

In a further embodiment, the invention relates to an assay or protein biochip for identifying and characterizing a substance for multiple sclerosis, characterized in that an arrangement or assay according to the invention a.) is brought in contact with at least one substance to be analyzed and b.) successful binding is detected.

The invention further relates to a method for identifying and characterizing a substance for multiple sclerosis, characterized in that an arrangement or assay according to the invention a.) is brought in contact with at least one substance to be analyzed and b.) successful binding is detected.

The substance to be analyzed can be any arbitrary native or non-native biomolecule, a synthetic chemical molecule, a mixture, or a substance library.

After the substance to be analyzed has come in contact with a marker sequence, the successful binding process is evaluated, which can take place, for example, using commercially available image analysis software (GenePix Pro (Axon Laboratores), Aida (Raytest), ScanArray (Packard Bioscience)).

The protein-protein interactions according to the invention (for example protein on marker sequence, such as antigen/antibody) or corresponding “means for detecting successful binding” can be visualized, for example, in the customary manner by way of fluorescent labeling, biotinylation, radioisotope labeling or colloidal gold or latex particle labeling. Bound antibodies are detected with the aid of secondary antibodies labeled with commercially available reporter molecules (for example Cy, Alexa, Dyomics, FITC or similar fluorescent dyes, colloidal gold or latex particles), or with reporter enzymes, such as alkaline phosphatase, horseradish peroxidase, or the like, and the corresponding colorimetric, fluorescent or chemiluminescent substrates. Readout is carried out, for example, by way of a microarray laser scanner, a CCD camera or visually.

In a further embodiment, the invention relates to a pharmaceutical/active ingredient or prodrug developed for multiple sclerosis and obtainable through the use of the assay or protein biochip according to the invention.

The invention therefore likewise relates to the use of an arrangement according to the invention or an assay for screening active ingredients for multiple sclerosis.

In a further embodiment, the invention therefore likewise relates to a target for the treatment and therapy of multiple sclerosis, selected in each case from the group SEQ 1-308 or a protein coding therefor.

In a further embodiment, the invention likewise relates to the use of the marker sequences according to the invention, preferably in the form of an arrangement as an affinity material for carrying out apheresis or, in the broader sense, dialysis, wherein substances from body fluids of a patient with multiple sclerosis, such as blood or plasma, bind to the marker sequences according to the invention and consequently can be selectively withdrawn from the body fluid.

### EXAMPLES AND FIGURES

Ten or more patient samples were individually screened against a cDNA expression library. The multiple sclerosis-specific expression clones were determined by way of comparison to ten or more healthy samples. The identity of the marker sequences was determined by way of DNA sequencing.

FIG. 1 shows the differential screening between two protein biochips from a cDNA expression library of a patient and a healthy participant, respectively. The differential clones are detected by way of fluorescent labeling and evaluated by way of bioinformatics.

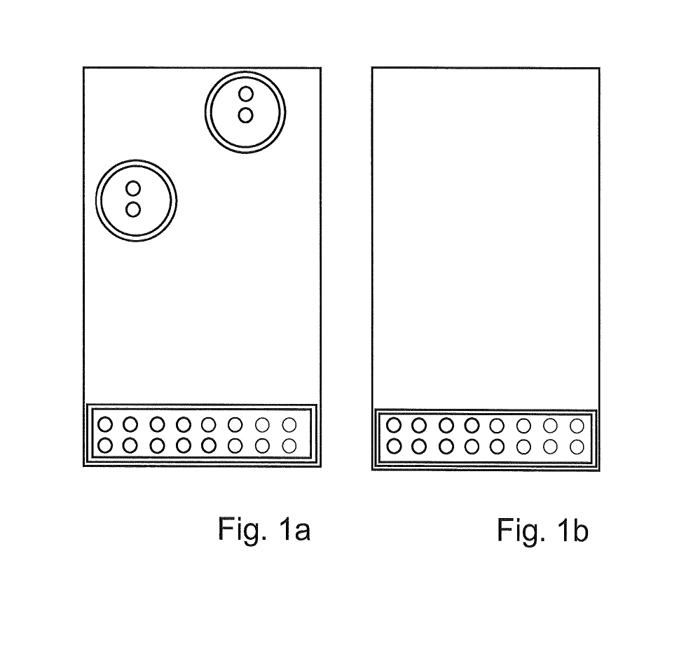

Within the scope of the biomarker identification, various bioinformatics analyses are carried out. Reactivities against approximately 2000 different antigens are measured for each serum using microarrays. This data is used to rank the spotted antigens with respect to the differentiation capability thereof between healthy and diseased sera. This evaluation is carried out by way of the non-parametric Mann-Whitney tests using normalized intensity data. An internal standard, which is also spotted on each chip, is used for normalization purposes. Because a p-value is calculated for each antigen, methods for correcting multiple testing are employed. A very conservative approach that is taken is to carry out a Bonferroni correction, and additionally the less restrictive false discovery rate (FDR) according to Benjamini & Hochberg is calculated.

Additionally, the data is utilized to classify the sera. To this end, different multivariate methods are employed. These are methods selected from statistical learning methods such as support vector machines (SVM), neuronal networks or classification trees, as well as a threshold value method, which is suitable both for classifying and for visually representing the data.

So as to avoid overfitting, tenfold cross validation of the data is carried out.

TABLE A
(gi accession number valid as of Oct. 1, 2010)

| SEQ 1b-308b | SEQ 1a-308a | |
|---|---|---|
| gi Acc Protein | gi Acc cDNA | NAME |
| gi\|157266266 | gi\|157266265 | WD repeat-containing protein 86 [Homo sapiens] |
| gi\|63252910 | gi 63252909 | DDB1- and CUL4-associated factor 6 isoform b |
| | | [Homo sapiens] |
| gi\|18699734 | gi\|20357519 | uridine-cytidine kinase 2 [Homo sapiens] |
| gi\|4505677 | gi\|260436978 | calcium/calmodulin-dependent 3′,5′-cyclic |
| | | nucleotide phosphodiesterase 1B isoform 1 |
| | | [Homo sapiens] |
| gi\|51477716 | gi\|51477715 | alpha-mannosidase 2x [Homo sapiens] |
| gi\|24797097 | gi\|24797096 | pyrroline-5-carboxylate reductase 1, mitochondrial |
| | | isoform 1 [Homo sapiens] |
| gi\|61676188 | gi\|195963314 | E3 ubiquitin-protein ligase HUWE1 [Homo sapiens] |
| gi\|30795119 | gi\|30795118 | F-box only protein 18 isoform 2 [Homo sapiens] |
| gi\|33469964 | gi\|33469963 | splicing factor 4 [Homo sapiens] |
| gi\|83035136 | gi\|217272875 | F-box only protein 31 [Homo sapiens] |
| gi\|6005747 | gi\|54792140 | E3 ubiquitin-protein ligase RING2 [Homo sapiens] |
| gi\|33636722 | gi\|261278365 | lipid phosphate phosphatase-related protein type 4 |
| | | isoform 1 [Homo sapiens] |
| gi\|145199237 | gi\|145199236 | RNA exonuclease 1 homolog [Homo sapiens] |
| gi\|24308201 | gi\|41327713 | adipocyte membrane-associated protein |
| | | [Homo sapiens] |
| gi\|33356547 | gi\|33356546 | DNA replication licensing factor MCM2 |
| | | [Homo sapiens] |
| gi\|7662074 | gi\|209413745 | zinc finger and BTB domain-containing protein 5 |
| | | [Homo sapiens] |
| gi\|6005924 | gi\|115583673 | downregulated in renal cell carcinoma |
| | | [Homo sapiens] |
| gi\|4505685 | gi\|291084749 | pyruvate dehydrogenase E1 component subunit |
| | | alpha, somatic form, mitochondrial isoform 1 |
| | | precursor [Homo sapiens] |
| gi\|20070228 | gi\|39725676 | nucleobindin-1 precursor [Homo sapiens] |
| gi\|21700763 | gi\|46361989 | hematological and neurological expressed 1-like |
| | | protein [Homo sapiens] |
| gi\|19923927 | gi\|221139831 | general transcription factor 3C polypeptide 6 |
| | | [Homo sapiens] |
| gi\|26051235 | gi\|26051234 | nuclear pore complex protein Nup133 [Homo sapiens] |
| gi\|50593021 | gi\|50593020 | NFU1 iron-sulfur cluster scaffold homolog, |
| | | mitochondrial isoform 2 [Homo sapiens] |
| gi\|38454194 | gi\|38454193 | gamma-tubulin complex component 4 [Homo sapiens] |
| gi\|5902122 | gi\|197381953 | spectrin beta chain, brain 2 [Homo sapiens] |
| gi\|62739181 | gi\|62739180 | rhotekin isoform c [Homo sapiens] |
| gi\|215599981 | gi\|215599980 | cyclin-D-binding Myb-like transcription factor 1 |
| | | isoform b [Homo sapiens] |
| gi\|134288890 | gi\|148596939 | DIS3-like exonuclease 2 [Homo sapiens] |
| gi\|32698750 | gi\|32698749 | SR-related CTD-associated factor 1 [Homo sapiens] |
| gi\|4501887 | gi\|11038618 | actin, cytoplasmic 2 [Homo sapiens] |
| gi\|4758112 | gi\|93588182 | spliceosome RNA helicase BAT1 [Homo sapiens] |
| gi\|58331179 | gi\|58331178 | rho GTPase-activating protein 39 [Homo sapiens] |
| gi\|27477111 | gi\|119393888 | pre-mRNA-splicing factor SLU7 [Homo sapiens] |
| gi\|145309326 | gi\|145309325 | laminin subunit gamma-1 precursor [Homo sapiens] |
| gi\|4507145 | gi\|23111044 | sorting nexin-4 [Homo sapiens] |
| gi\|284055255 | gi\|284055254 | CM P-N-acetylneuraminate-beta-1,4-galactoside |
| | | alpha-2,3-sialyltransferase isoform a |
| gi\|33356547 | gi\|33356546 | DNA replication licensing factor MCM2 |
| | | [Homo sapiens] |
| gi\|13259508 | gi\|13259507 | dynactin 1 isoform 2 [Homo sapiens] |
| gi\|24797103 | gi\|24797102 | RAS guanyl releasing protein 2 [Homo sapiens] |
| gi\|20070228 | gi\|39725676 | nucleobindin 1 [Homo sapiens] |
| gi\|4502101 | gi\|4502100 | annexin I [Homo sapiens] |
| gi\|16975484 | gi\|92091602 | centaurin delta 2 isoform b [Homo sapiens] |
| gi\|21707902 | gi\|21707901 | CTTN protein [Homo sapiens] |
| gi\|29788785 | gi\|34222261 | tubulin, beta [Homo sapiens] |
| gi\|28827795 | gi\|40549398 | charged multivesicular body protein 4b |
| | | [Homo sapiens] |
| gi\|149363636 | gi\|149363635 | plexin-B2 precursor [Homo sapiens] |
| gi\|66346681 | gi\|66346680 | plasminogen activator inhibitor 1 RNA-binding |
| | | protein isoform 2 [Homo sapiens] |
| gi\|40548422 | gi\|40548421 | charged multivesicular body protein 4a |
| | | [Homo sapiens] |
| gi\|3005715 | gi\|3005714 | protein 4.1-G [Homo sapiens] |
| gi\|22035672 | gi\|87196331 | thioredoxin reductase 2 precursor [Homo sapiens] |
| gi\|4506723 | gi\|70609888 | ribosomal protein S3a [Homo sapiens] |
| gi\|34485727 | gi\|153792638 | NCK-associated protein 1-like [Homo sapiens] |
| gi\|6912602 | gi\|38569401 | arfaptin-2 [Homo sapiens] |
| gi\|4506685 | gi\|14591910 | 40S ribosomal protein S13 [Homo sapiens] |
| gi\|18104948 | gi\|78190465 | 60S ribosomal protein L21 [Homo sapiens] |
| gi\|167466201 | gi\|167466200 | WAS protein family homolog 1 [Homo sapiens] |
| gi\|24234688 | gi\|156071496 | stress-70 protein, mitochondrial precursor |
| | | [Homo sapiens] |
| gi\|307574659 | gi\|307574658 | hypothetical protein LOC116328 isoform 2 |
| | | [Homo sapiens] |
| gi\|4506619 | gi\|78190466 | 60S ribosomal protein L24 [Homo sapiens] |
| gi\|94536842 | gi\|94536841 | ribose 5-phosphate isomerase A [Homo sapiens] |
| gi\|4505753 | gi\|31543395 | phosphoglycerate mutase 1 [Homo sapiens] |
| gi\|11545918 | gi\|210147465 | tubulointerstitial nephritis antigen-like 1 |
| | | [Homo sapiens] |
| gi\|163644321 | gi\|163644320 | cytochrome b-c1 complex subunit Rieske, |
| | | mitochondrial [Homo sapiens] |
| gi\|34526674 | gi\|34526673 | unnamed protein product [Homo sapiens] |
| gi\|4505573 | gi\|166064031 | rho guanine nucleotide exchange factor 7 |
| | | isoform a [Homo sapiens] |
| gi\|5902122 | gi\|197381953 | spectrin beta chain, brain 2 [Homo sapiens] |
| gi\|23618848 | gi\|56676380 | protein SYS1 homolog isoform a [Homo sapiens] |
| gi\|83641870 | gi\|262331549 | nucleophosmin isoform 3 [Homo sapiens] |
| gi\|54112429 | gi\|54112428 | dedicator of cytokinesis protein 7 [Homo sapiens] |
| gi\|5453690 | gi\|5453689 | dnaJ homolog subfamily B member 1 [Homo sapiens] |
| gi\|10863945 | gi\|195963391 | X-ray repair cross-complementing protein 5 |
| | | [Homo sapiens] |
| gi\|16753215 | gi\|94538348 | profilin-2 isoform a [Homo sapiens] |
| gi\|149363636 | gi\|149363635 | plexin-B2 precursor [Homo sapiens] |
| gi\|27501458 | gi\|157951660 | chromosome transmission fidelity protein 18 |
| | | homolog [Homo sapiens] |
| gi\|205277463 | gi\|306518580 | transketolase [Homo sapiens] |
| gi\|21396500 | gi\|300116295 | HIRA interacting protein 3 [Homo sapiens] |
| gi\|71565154 | gi\|71565153 | alcohol dehydrogenase class-3 [Homo sapiens] |
| gi\|34147630 | gi\|169658370 | elongation factor Tu, mitochondrial precursor |
| | | [Homo sapiens] |
| gi\|5729875 | gi\|216547928 | membrane-associated progesterone receptor |
| | | component 1 [Homo sapiens] |
| gi\|50592996 | gi\|50592995 | tubulin beta-3 chain [Homo sapiens] |
| gi\|5802966 | gi\|58530846 | destrin isoform a [Homo sapiens] |
| gi\|19923315 | gi\|261862340 | serine hydroxymethyltransferase, mitochondrial |
| | | isoform 1 precursor [Homo sapiens] |
| gi\|31982933 | gi\|33946335 | DNA-binding protein inhibitor ID-2 [Homo sapiens] |
| gi\|4506713 | gi\|294459919 | ubiquitin-40S ribosomal protein S27a precursor |
| | | [Homo sapiens] |
| gi\|16753227 | gi\|67189547 | 60S ribosomal protein L6 [Homo sapiens] |
| gi\|50592996 | gi\|50592995 | tubulin beta-3 chain [Homo sapiens] |
| gi\|55743075 | gi\|96322659 | angio-associated migratory cell protein |
| | | [Homo sapiens] |
| gi\|13569962 | gi\|116014337 | ras-related protein Rab-1B [Homo sapiens] |
| gi\|78395056 | gi\|78395055 | C15orf23 protein [Homo sapiens] |
| gi\|45439359 | gi\|45439358 | triple functional domain protein [Homo sapiens] |
| gi\|34335253 | gi\|254911094 | disks large-associated protein 4 isoform a |
| | | [Homo sapiens] |
| gi\|34098946 | gi\|109134359 | nuclease-sensitive element-binding protein 1 |
| | | [Homo sapiens] |
| gi\|195972909 | gi\|195972908 | nasal embryonic luteinizing hormone-releasing |
| | | Homo sapiens isoform a [Homo sapiens] |
| gi\|8394499 | gi\|283945567 | ubiquitin-associated protein 1 isoform 1 |
| | | [Homo sapiens] |
| gi\|24234688 | gi\|296080701 | stress-70 protein, mitochondrial precursor |
| | | [Homo sapiens] |
| gi\|4758138 | gi\|221139768 | probable ATP-dependent RNA helicase DDX5 |
| | | [Homo sapiens] |
| gi\|34335251 | gi\|109891935 | disks large-associated protein 4 isoform b |
| | | [Homo sapiens] |
| gi\|11342680 | gi\|197245402 | beta-centractin [Homo sapiens] |
| gi\|5453832 | gi\|169234641 | hypoxia up-regulated protein 1 precursor |
| | | [Homo sapiens |
| gi\|34098946 | gi\|109134359 | nuclease-sensitive element-binding protein 1 |
| | | [Homo sapiens] |
| gi\|154090959 | gi\|154090958 | WASH complex subunit FAM21B [Homo sapiens] |
| gi\|4506913 | gi\|209693454 | beta-sarcoglycan [Homo sapiens] |
| gi\|247425318 | gi\|247425317 | immunoglobulin heavy chain variable region |
| | | [Homo sapiens] |
| gi\|5031701 | gi\|95104789 | follistatin-like 3 (secreted glycoprotein) |
| gi\|13375616 | gi\|34304362 | fatty acid desaturase 3 [Homo sapiens] |
| gi\|13376888 | gi\|34147389 | transmembrane protein 121 [Homo sapiens] |
| gi\|17986283 | gi\|17986282 | tubulin alpha-1A chain [Homo sapiens] |
| gi\|5031669 | gi\|39725675 | cyclin-dependent kinase 2-associated protein 2 |
| | | [Homo sapiens] |
| gi\|14249132 | gi\|270309185 | protein BEX2 isoform 3 [Homo sapiens] |
| gi\|22027541 | gi\|22027540 | programmed cell death protein 7 [Homo sapiens] |
| gi\|4507761 | gi\|77539056 | ubiquitin-60S ribosomal protein L40 precursor |
| | | [Homo sapiens] |
| gi\|19353009 | gi\|19353008 | Similar to Elongation factor 2b [Homo sapiens] |
| gi\|110815842 | gi\|34147354 | dysbindin domain-containing protein 1 isoform 2 |
| | | [Homo sapiens] |
| gi\|171846268 | gi\|291084495 | elongation factor Ts, mitochondrial isoform 2 |
| | | precursor [Homo sapiens] |
| gi\|4557325 | gi\|48762938 | apolipoprotein E precursor [Homo sapiens] |
| gi\|41406055 | gi\|228008404 | amyloid beta A4 protein isoform b precursor |
| | | [Homo sapiens]. |
| gi\|23510421 | gi\|23510420 | tumor necrosis factor receptor superfamily; member |
| | | 6 isoform 2 precursor [Homo sapiens] |
| gi\|15718706 | gi\|122056469 | caspase 8 isoform B precursor [Homo sapiens] |
| gi\|119575060 | | Homo sapiens CD24 molecule (CD24) |
| gi\|17978489 | gi\|68508947 | Homo sapiens CD97 molecule (CD97); transcript |
| | | variant 1 |
| gi\|52630326 | gi\|158420733 | chromodomain helicase DNA binding protein 3 |
| | | [Homo sapiens] |
| gi\|4503481 | gi\|83656774 | eukaryotic translation elongation factor 1 gamma |
| | | [Homo sapiens] |
| gi\|116063573 | gi\|160420313 | filamin A; alpha isoform 1 [Homo sapiens]. |
| gi\|4503979 | gi\|196115280 | glial fibrillary acidic protein isoform 1 |
| | | [Homo sapiens] |
| gi\|150418002 | gi\|150418001 | HLA class II histocompatibility antigen, DQ beta 1 |
| | | chain precursor [Homo sapiens] |
| gi\|55925614 | gi\|55925613 | interferon alpha-inducible protein 27, mitochondrial |
| | | isoform 2 [Homo sapiens] |
| gi\|10835145 | gi\|27894305 | interleukin 1; beta proprotein [Homo sapiens] |
| gi\|28610151 | gi\|28610150 | interleukin-7 receptor subunit alpha precursor |
| | | [Homo sapiens] |
| gi\|119703755 | gi\|119703754 | laminin; beta 2 precursor [Homo sapiens]. |
| gi\|5031877 | gi\|27436949 | Lamin B1 [Homo sapiens] |
| gi\|82534351 | gi\|189409155 | microtubule-associated protein tau isoform 1 |
| gi\|4505241 | gi\|98991774 | protein Mpv17 [Homo sapiens] |
| gi\|222136617 | gi\|222136616 | interferon-induced GTP-binding protein Mx1 |
| | | [Homo sapiens] |
| gi\|105990539 | gi\|197927150 | neurofilament; light polypeptide 68 kDa |
| | | [Homo sapiens] |
| gi\|27886561 | gi\|27886560 | nuclear factor of activated T-cells, cytoplasmic 3 |
| | | isoform 1 [Homo sapiens] |
| gi\|205360954 | gi\|205360953 | polycystin-1 isoform 1 precursor [Homo sapiens] |
| gi\|32171249 | gi\|38505192 | prostaglandin-H2 D-isomerase [Homo sapiens] |
| gi\|18641347 | gi\|115385975 | Homo sapiens protein tyrosine phosphatase; |
| | | receptor type; C (PTPRC); transcript variant 1 |
| gi\|4506367 | gi\|209915550 | ras-related protein Rab-3A [Homo sapiens] |
| gi\|4506701 | gi\|71772514 | 40S ribosomal protein S23 [Homo sapiens] |
| gi\|4506841 | gi\|56119169 | C-C motif Chemokine 2 precursor [Homo sapiens] |
| gi\|19557702 | gi\|157266286 | surfeit 6 [Homo sapiens] |
| gi\|21359969 | gi\|141801742 | DNA-directed RNA polymerase III subunit RPC3 |
| | | [Homo sapiens] |
| gi\|5803227 | gi\|21464103 | 14-3-3 protein theta [Homo sapiens] |
| gi\|148747351 | gi\|296841089 | protein kinase C and casein kinase substrate in |
| | | neurons 2 [Homo sapiens] |
| gi\|11321634 | gi\|125987597 | CD2-associated protein [Homo sapiens] |
| gi\|223468620 | gi\|223468619 | E3 ubiquitin-protein ligase makorin-1 isoform 1 |
| | | [Homo sapiens] |
| gi\|24308113 | gi\|291190751 | KIF1 binding protein [Homo sapiens]. |
| gi\|82617630 | gi\|82617629 | Cytoplasmic FMR1 interacting protein 2 |
| | | [Homo sapiens] |
| gi\|48949851 | gi\|48949850 | HERV-W_7q21.2 provirus ancestral Env polyprotein |
| | | precursor [Homo sapiens] |
| gi\|7706702 | gi\|28144902 | interleukin-23 subunit alpha precursor |
| | | [Homo sapiens] |
| gi\|115299754 | gi\|115299753 | dysbindin domain-containing protein 2 isoform b |
| | | [Homo sapiens] |
| gi\|70887780 | gi\|70887779 | Homo sapiens sperm associated antigen 16 |
| gi\|119120897 | gi\|119120896 | partitioning defective 3 homolog B isoform b |
| | | [Homo sapiens] |
| gi\|209969690 | gi\|209969689 | hypothetical protein LOC119032 [Homo sapiens] |
| gi\|187960086 | gi\|187960086 | cytochrome P450 4V2 [Homo sapiens] |
| gi\|6978649 | gi\|242246959 | choline/ethanolamine kinase [Homo sapiens] |
| gi\|4506649 | gi\|76496470 | 60S ribosomal protein L3 isoform a [Homo sapiens] |
| gi\|27545326 | gi\|55956799 | SWI/SNF related, matrix associated, actin |
| | | dependent regulator of chromatin, subfamily b, |
| | | member 1 [Homo sapients] |
| gil\|15527080 | gi\|115527079 | metastasis-associated protein MTA1 [Homo sapiens] |
| gi\|56788399 | gi\|56788398 | GI: 56788398 |
| gi\|239787092 | gi\|239787091 | TNFAIP3-interacting protein 2 isoform 1 |
| | | [Homo sapiens] |
| gi\|54112429 | gi\|54112428 | dedicator of cytokinesis protein 7 [Homo sapiens] |
| gi\|4504603 | gi\|50593016 | Interferon beta1 |
| gi\|4504605 | gi\|4504604 | Interferon omega |
| gi\|10834984 | gi\|224831235 | Interleukin IL-6 |
| gi\|10835141 | gi\|24430216 | Interleukin IL-10 |
| gi\|24430219 | gi\|24430218 | interleukin-12 subunit alpha precursor |
| | | [Homo sapiens] |
| gi\|24497438 | gi\|24497437 | interleukin-12 subunit beta precursor |
| | | [Homo sapiens] |
| gi\|25306235 | gi\|219842281 | brain-derived neurotrophic factor isoform b |
| | | preproprotein [Homo sapiens] |
| gi\|4758020 | gi\|209574322 | ciliary neurotrophic factor [Homo sapiens] |
| gi\|10834978 | gi\|28610153 | interleukin-8 precursor [Homo sapiens] |
| gi\|89903008 | gi\|237858673 | neurofascin isoform 4 precursor [Homo sapiens] |
| gi\|17158044 | gi\|17158043 | ribosomal protein S6 [Homo sapiens] |
| gi\|51476647 | gi\|51476646 | Ankyrin repeat and SAM domain-containing protein 6 |
| gi\|72534660 | gi\|197209865 | splicing factor, arginine/serine-rich 7 [Homo sapiens] |
| gi\|13128968 | gi\|13128967 | dual specificity phosphatase 26 [Homo sapiens] |
| gi\|22538467 | gi\|22538466 | proteasome (prosome, macropain) subunit, beta |
| | | type, 4 [Homo sapiens] |
| gi\|15055539 | gi\|70609878 | ribosomal protein S2 [Homo sapiens] |
| gi\|3129006 | gi\|13129005 | DEAD (Asp-Glu-Ala-Asp) box polypeptide 50 |
| | | [Homo sapiens] |
| gi\|4506663 | gi\|72377361 | ribosomal protein L8 [Homo sapiens] |
| gi\|19923193 | gi\|21237722 | suppression of tumorigenicity 13 (colon carcinoma) |
| | | (Hsp70 interacting protein) (ST13) |
| gi\|4506649 | gi\|76496470 | ribosomal protein L3 isoform a [Homo sapiens] |
| gi\|4506743 | gi\|4506742 | ribosomal protein S8 [Homo sapiens] |
| gi\|5031877 | gi\|27436949 | lamin B1 [Homo sapiens] |
| gi\|94536842 | gi\|94536841 | ribose 5-phosphate isomerase A [Homo sapiens] |
| gi\|17157993 | gi\|141803509 | olfactomedin 2 [Homo sapiens] |
| gi\|8922911 | gi\|8922910 | radical S-adenosyl methionine domain containing 1 |
| | | [Homo sapiens] |
| gi\|4826724 | gi\|17105402 | zygin 1 isoform 1 [Homo sapiens] |
| gi\|6806913 | gi\|187960101 | centaurin, alpha 1 [Homo sapiens] |
| gi\|5454058 | gi\|5454057 | ST3 beta-galactoside alpha-2,3-sialyltransferase 4 |
| | | [Homo sapiens] |
| gi\|54607091 | gi\|54607090 | SUMO1/sentrin/SMT3 specific protease 2 [Homo sapiens] |
| gi\|166063995 | gi\|166063994 | general transcription factor IIIA [Homo sapiens] |
| gi\|72534684 | gi\|166197669 | phospholipase D family, member 3 [Homo sapiens] |
| gi\|14591909 | gi\|71772259 | ribosomal protein L5 [Homo sapiens] |
| gi\|114l5026 | gi\|15431299 | ribosomal protein L18a [Homo sapiens] |
| gi\|13129004 | gi\|30089943 | GIY-YIG domain containing 2 isoform 1 [Homo sapiens] |
| gi\|62414289 | gi\|240849334 | vimentin [Homo sapiens] |
| gi\|110347461 | gi\|110347460 | MYC-associated zinc finger protein isoform 1 |
| | | [Homo sapiens] |
| gi\|4758648 | gi\|187761329 | kinesin family member 5B [Homo sapiens] |
| gi\|4502337 | gi\|38372939 | alpha-2-glycoprotein 1, zinc-binding [Homo sapiens] |
| gi\|6912642 | gi\|142383813 | sex comb on midleg 1 isoform 2 [Homo sapiens] |
| gi\|4503065 | gi\|62241006 | crystallin, mu isoform 1 [Homo sapiens] |
| gi\|4505409 | gi\|66392201 | nucleoside diphosphate kinase B [Homo sapiens] |
| gi\|4507791 | gi\|150417997 | ubiquitin-conjugating enzyme E2M (UBC12 |
| | | homolog, yeast) |
| gi\|7657015 | gi\|187936926 | hypothetical protein LOC51493 [Homo sapiens] |
| gi\|16554609 | gi\|16554608 | 28S ribosomal protein S11, mitochondrial isoform a |
| | | [Homo sapiens] |
| gi\|29725611 | gi\|30065641 | serine/threonine-protein phosphatase 2A activator |
| | | isoform b [Homo sapiens] |
| gi\|5902082 | gi\|151101481 | ST3 beta-galactoside alpha-2,3-sialyltransferase 2 |
| | | [Homo sapiens] |
| gi\|29893564 | gi\|34222264 | microspherule protein 1 isoform 1 [Homo sapiens] |
| gi\|19923796 | gi\|142359942 | 60S ribosomal export protein NMD3 [Homo sapiens] |
| gi\|4506661 | gi\|18390348 | ribosomal protein L7a [Homo sapiens] |
| gi\|16579885 | gi\|16579884 | 60S ribosomal protein L4 [Homo sapiens] |
| gi\|40548389 | gi\|66346686 | dickkopf homolog 3 precursor [Homo sapiens] |
| gi\|5453880 | gi\|221219065 | acidic (leucine-rich) nuclear phosphoprotein 32 |
| | | family, member A [Homo sapiens] |
| gi\|22035558 | gi\|48833509 | transcription factor IIIB 90 kDa subunit isoform 3 |
| | | [Homo sapiens] |
| gi\|4506617 | gi\|78000184 | ribosomal protein L17 [Homo sapiens] |
| gi\|27545323 | gi\|168229166 | chondroitin polymerizing factor [Homo sapiens] |
| gi\|62750347 | gi\|62750346 | histone deacetylase 5 isoform 1 [Homo sapiens] |
| gi\|100913206 | gi\|00913205 | ATP-dependent RNA helicase A [Homo sapiens] |
| gi\|106879210 | gi\|106879209 | SH2 domain-containing adapter protein B |
| | | [Homo sapiens] |
| gi\|18152783 | gi\|18490985 | 60S ribosomal protein L10-like [Homo sapiens] |
| gi\|15431301 | gi\|72187675 | ribosomal protein L7 [Homo sapiens] |
| gi\|13375618 | gi\|114155130 | 24-dehydrocholesterol reductase precursor |
| | | [Homo sapiens] |
| gi\|14043070 | gi\|83641894 | heterogeneous nuclear ribonucleoprotein A1 |
| | | [Homo sapiens] |
| gi\|22202633 | gi\|88999578 | prefoldin subunit 5 isoform alpha [Homo sapiens] |
| gi\|22907052 | gi\|300360513 | actin-related protein 2/3 complex subunit 1A isoform |
| | | 1 [Homo sapiens] |
| gi\|23308577 | gi\|217272837 | D-3-phosphoglycerate dehydrogenase [Homo sapiens] |
| gi\|34098946 | gi\|109134359 | nuclease-sensitive element-binding protein 1 |
| | | [Homo sapiens] |
| gi\|4502015 | gi\|109637793 | A-kinase anchor protein 1 precursor [Homo sapiens] |
| gi\|4502027 | gi\|215982788 | albumin preproprotein [Homo sapiens] |
| gi\|4506631 | gi\|15812218 | 60S ribosomal protein L30 [Homo sapiens] |
| gi\|4506667 | gi\|49087144 | 60S acidic ribosomal protein P0 Homo sapiens] |
| gi\|4508007 | gi\|197382778 | zinc finger protein 174 isoform a [Homo sapiens] |
| gi\|5031851 | gi\|44889961 | stathmin isoform a [Homo sapiens] |
| gi\|52630322 | gi\|58420732 | chromodomain-helicase-DNA-binding protein 3 |
| | | isoform 2 [Homo sapiens] |
| gi\|87080813 | gi\|87080812 | LON peptidase N-terminal domain and ring finger 1 |
| | | [Homo sapiens] |
| gi\|9945439 | gi\|90193629 | septin-5 [Homo sapiens] |
| gi\|5803013 | gi\|77628146 | endoplasmic reticulum protein 29 isoform 1 |
| | | precursor [Homo sapiens] |
| gi\|168229248 | gi\|168229247 | asparagine synthetase [Homo sapiens] |
| gi\|90652861 | gi\|90652860 | protein tyrosine phosphatase, non-receptor type 5 |
| | | (striatum-enriched) isoform b [Homo sapiens] |
| gi\|58761502 | gi\|58761501 | GTP-binding protein PTD004 isoform 2 [Homo sapiens] |
| gi\|94538370 | gi\|94538369 | DnaJ (Hsp40) homolog, subfamily C, member 2 |
| | | isoform 1 [Homo sapiens] |
| gi\|10047104 | gi\|110227859 | synovial sarcoma translocation gene on |
| | | chromosome 18-like 2 [Homo sapiens] |
| gi\|9951915 | gi\|239937553 | S-adenosylhomocysteine hydrolase [Homo sapiens] |
| gi\|4507729 | gi\|68299771 | tubulin, beta 2 [Homo sapiens] |
| gi\|5031875 | gi\|153281091 | lamin A/C isoform 2 [Homo sapiens] |
| gi\|41393561 | gi\|41393560 | leucine aminopeptidase 3 [Homo sapiens] |
| gi\|157885806 | gi\|157885805 | chromosome 12 open reading frame 51 [Homo sapiens] |
| gi\|119624431 | 0 | hCG2041192 [Homo sapiens] |
| gil\|12382250 | gi\|112382249 | spectrin, beta, non-erythrocytic 1 isoform 1 |
| | | [Homo sapiens] |
| gi\|93141029 | gi\|93141028 | paralemmin isoform 2 [Homo sapiens] |
| gi\|4501867 | gi\|46411160 | aconitase 2 precursor [Homo sapiens] |
| gi\|88758580 | gi\|221554513 | cyclin L2 isoform A [Homo sapiens] |
| gi\|5901922 | gi\|39995072 | cell division cycle 37 protein [Homo sapiens] |
| gi\|21624607 | gi\|23510452 | coactosin-like 1 [Homo sapiens] |
| gi\|24234747 | gi\|24234746 | interleukin enhancer binding factor 2 [Homo sapiens] |
| gi\|160420328 | gi\|160420327 | iron-sulfur cluster assembly 2 [Homo sapiens] |
| gi\|6005743 | gi\|62241020 | DEAD (Asp-Glu-Ala-As) box polypeptide 19 isoform |
| | | 1 [Homo sapiens] |
| gi\|15010818 | gi\|15010817 | JKTBP1delta6 [Homo sapiens] |
| gi\|4502847 | gi\|186972139 | cold inducible RNA binding protein [Homo sapiens] |
| gi\|71164894 | gi\|71164893 | trinucleotide repeat containing 4, isoform CRA_a |
| | | [Homo sapiens] |
| gi\|4758206 | gi\|187608704 | dual specificity phosphatase 2 [Homo sapiens] |
| gi\|5730009 | gi\|115387097 | ret finger protein [Homo sapiens] |
| gi\|7657689 | gi\|21327683 | YME1-like 1 isoform 3 [Homo sapiens] |
| gi\|28872792 | gi\|28872791 | CDK5 regulatory subunit associated protein 3 |
| | | [Homo sapiens] |
| gi\|15147333 | gi\|189458899 | tripartite motif-containing 37 protein [Homo sapiens] |
| gi\|29826319 | gi\|29826318 | adducin 1 (alpha) isoform a [Homo sapiens] |
| gi\|190194416 | gi\|190194415 | F-box and leucine-rich repeat protein 15 |
| | | [Homo sapiens] |
| gi\|4758272 | gi\|46389548 | endosulfine alpha isoform 3 [Homo sapiens] |
| gi\|166795250 | gi\|166795249 | kinesin family member 2C [Homo sapiens] |
| gi\|151301215 | gi\|151301214 | widely-interspaced zinc finger motifs [Homo sapiens] |
| gi\|46048234 | gi\|194294559 | nucleolar protein 8 [Homo sapiens] |
| gi\|219842250 | gi\|219842249 | periphilin 1 isoform 8 [Homo sapiens] |
| gi\|48762942 | gi\|48762941 | huntingtin interacting protein-1-related |
| | | [Homo sapiens] |
| gi4506003 | gi\|45827796 | protein phosphatase 1, catalytic subunit, alpha |
| | | isoform 1 [Homo sapiens] |
| gi\|67782338 | gi\|67782337 | amyloid precursor-like protein 1 isoform 1 precursor |
| | | [Homo sapiens] |
| gi\|17402896 | gi\|17402895 | RAD51 homolog C isoform 1 [Homo sapiens] |
| gi\|5453629 | gi\|34335254 | dynactin 2 [Homo sapiens] |
| gi\|11968182 | gi\|14165467 | ribosomal protein S18 [Homo sapiens] |
| gi\|122937289 | gi\|122937288 | kinesin family member 18B [Homo sapiens] |
| gi\|155029542 | gi\|155029541 | BEN domain containing 7 isoform 1 [Homo sapiens] |
| gi\|11321585 | gi\|20357526 | guanine nucleotide-binding protein, beta-1 subunit |
| | | [Homo sapiens] |
| gi\|12597635 | gi\|12597634 | B-cell CLL/lymphoma 11B isoform 2 [Homo sapiens] |
| gi\|4502751 | gi\|17981697 | cyclin-dependent kinase inhibitor 2C (p18, inhibits |
| | | CDK4), isoform CRA_b [Homo sapiens] |
| gi\|7662046 | gi\|7662045 | myeloid/lymphoid or mixed-lineage leukemia 4 |
| | | [Homo sapiens] |
| gi\|37537687 | gi\|37537686 | zinc finger protein 444 [Homo sapiens] |
| gi\|133922582 | gi\|133922581 | zinc finger protein 358 [Homo sapiens] |
| gi\|189083826 | gi\|189083825 | inhibitor of growth family, member 4 isoform 3 |
| | | [Homo sapiens] |
| gi\|110224479 | gi\|110224478 | prosaposin isoform c preproprotein [Homo sapiens] |
| gi\|21264343 | gi\|21264342 | scaffold attachment factor B [Homo sapiens] |
| gi\|14670375 | gi\|14670374 | SCG10-like-protein [Homo sapiens] |
| gi\|12545395 | gi\|94721348 | islet cell autoantigen 1 [Homo sapiens] |
| gi\|91199552 | gi\|91199551 | ADP-ribosylation factor-like protein 16 |
| | | [Homo sapiens] |
| gi\|21614499 | gi\|161702984 | ezrin [Homo sapiens] |
| gi\|5453690 | gi\|5453689 | DnaJ (Hsp40) homolog, subfamily B, member 1 |
| | | [Homo sapiens] |
| gi\|20070228 | gi\|39725676 | nucleobindin 1 [Homo sapiens] |
| gi\|12667788 | gi\|225703132 | myosin, heavy polypeptide 9, non-muscle |
| | | [Homo sapiens] |
| gi\|5902158 | gi\|215422343 | ring finger protein 113A [Homo sapiens] |
| gi\|62241011 | gi\|62241010 | v-akt murine thymoma viral oncogene homolog 1 |
| | | [Homo sapiens] |
| gi\|27734911 | gi\|27734910 | DAZ interacting protein 1-like [Homo sapiens] |
| gi\|7669492 | gi\|83641890 | glyceraldehyde-3-phosphate dehydrogenase |
| | | [Homo sapiens] |
| gi\|7657514 | gi\|21314660 | GTP-binding protein RHO6 [Homo sapiens] |
| gi\|38257139 | gi\|38257138 | protein kinase, cAMP-dependent, regulatory, type I, |
| | | beta [Homo sapiens] |
| gi\|23503295 | gi\|26787971 | casein kinase 2, beta polypeptide [Homo sapiens] |
| gi\|20128774 | gi\|47519746 | mitogen-activated protein kinase 11 [Homo sapiens] |
| gi\|4759274 | gi\|215422360 | thioredoxin-like 1 [Homo sapiens] |
| gi\|32189394 | gi\|50345985 | ATP synthase, H+ transporting, mitochondrial F1 |
| | | complex, beta subunit precursor [Homo sapiens] |

1-19. (canceled)

20. A method for diagnosing multiple sclerosis, comprising
a) contacting at least one marker sequence of a cDNA selected from the group consisting of SEQ 1-308 and SEQ 1a-308a, or a respective protein encoded thereby, or a respective partial sequence or fragment thereof, fixed on a solid support, with body fluid or tissue extract of a patient, and
b) detecting an interaction of the body fluid or tissue extract with the marker sequences from a).

21. The method of claim 20, wherein said at least one marker sequence is at least one protein encoded by a cDNA selected from the group consisting of SEQ 1-308 and SEQ 1a-308a, and said method further comprising normalizing said least one marker with autoantibodies from patients who do not have multiple sclerosis.

22. The method of claim 20, wherein said body fluid is obtained from cerebrospinal fluid of said patient.

22. A method for risk stratification, or for managing the treatment of a patient with multiple sclerosis, comprising determining at least one marker sequence of a cDNA selected from the group SEQ 1-308 and/or SEQ 1a-308, or a respective protein coding therefor, or a respective partial sequence or fragment thereof, from a patient.

23. The method of claim 7, wherein the stratification or the treatment management comprises decisions regarding the treatment and therapy of the patient, hospitalization of the patient, use, effect or dosage of one or more pharmaceuticals, a therapeutic measure, or monitoring the progression of an illness or treatment, etiology, or classification of a disease.

24. An arrangement of marker sequences comprising at least one marker sequence of a cDNA selected from the group consisting of SEQ 1-308 and SEQ 1a-308a, or a respective protein encoded thereby.

25. The arrangement of claim 24, wherein said arrangement comprises at least 2 to 5 or 10 marker sequences.

26. The arrangement of claim 24,wherein the marker sequences are present in the form of clones.

27. An assay, protein biochip comprising an arrangement according to claim 24, characterized in that the marker sequences are applied to a solid support.

28. A method for identifying and characterizing a substance for multiple sclerosis, comprising contacting an arrangement of claim 24 with at least one substance to be analyzed, and detecting binding of said at least one substance to a marker sequence of said arrangement.

29. A method for screening active ingredients for multiple sclerosis comprising contacting an arrangement of claim 24 with at least one substance to be analyzed, and detecting binding of said at least one substance to a marker sequence of said arrangement

30. A method for apheresis or dialysis for patients for multiple sclerosis comprising using an arrangement of claim 24 for carrying out apheresis or dialysis for patients with multiple sclerosis.
